(19) United States
(12) Patent Application Publication
(10) Pub. No.: US 2021/0330725 A1
(43) Pub. Date: Oct. 28, 2021

(54) GARCINIA MANGOSTANA EXTRACT FOR PROMOTING HAIR GROWTH

(71) Applicant: PIERRE FABRE DERMO-COSMETIQUE, Boulogne-Billancourt (FR)

(72) Inventors: Mathieu LETI, BAZIEGE (FR); Marguerite LEVEQUE, TOULOUSE (FR); Sylvie DAUNES-MARION, TOULOUSE (FR)

(21) Appl. No.: 17/311,097

(22) PCT Filed: Dec. 4, 2019
(86) PCT No.: PCT/FR2019/052925

§ 371 Date: Jun. 4, 2021

(30) Foreign Application Priority Data
Dec. 6, 2018 (FR) 1872420

(51) Int. Cl. A61K 36/38; A61K 8/9789; A61Q 7/00; A61P 17/14

(52) U.S. Cl. CPC A61K 36/38; A61K 8/9789; A61Q 7/00; A61K 2236/53; A61K 2236/333; A61K 2236/37; A61P 17/14

(57) ABSTRACT

The present invention relates to a garcinia mangostana extract and to compositions containing this extract for application in cosmetics and dermatology for promoting hair growth.

### TECHNICAL FIELD

The present invention relates to an extract of Garcinia mangostana L. and compositions containing this extract for application in the fields of cosmetics and dermatology to promote hair growth, more particularly to stimulate hair regrowth.

### PRIOR ART

Hair care, not only for cosmetic purposes but also to prevent hair loss and to regenerate hair, has always attracted research. Many theories have attempted to clarify the etiology of hair loss in cases of baldness, alopecia, etc., blaming it on seborrhea, increased tension of the tissue on the cranial sphere, reduced blood supply, or certain endocrine or nerve disorders.

The hair follicle is a mini-organ anchored in the skin to the hypodermis, the main function of which is the production of a hair shaft. Their distribution is established during growth in utero and their number is genetically determined. The hair follicle is a dynamic structure that produces hair during the growth and remodeling cycle of tissues. This cycle is divided into three phases:
A growth phase (anagen), the cells of the dermal papilla (fibroblasts) send a signal to the stem cells of the bulb which allows them to proliferate. Cells will transform and envelop the dermal papilla to form the hair's sulfur matrix. They divide and differentiate into follicular keratinocytes, the cells responsible for the structure of the hair. For the hair to be well structured, these keratinocytes need sulfur proteins, vitamin B6 and various minerals such as zinc and magnesium. The duration of this phase determines the length of the hair and depends on the proliferation and differentiation of the matrix cells at the base of the follicle.
A phase of regression (catagen), the matrix dies and thus the dermal papilla is no longer in contact with this matrix. There is no more exchange between the cells. The follicle and the dermal papilla go back up to the epidermis.
A resting phase (telogen), the cells of the dermal papilla and the bulb are intact and inactive. The hair falls out. For a new hair to develop, the cycle must be reinitiated.

The hair is therefore constantly being renewed and of the 100 000 to 150 000 hairs making up a head of hair, most are in the growth phase. There is a normal and physiological hair loss of about 60 to 100 per day for a healthy head of hair. Beyond that, hair loss is said to be pathological, whether it is occasional or permanent.

The term alopecia refers to the partial or general loss of hair. Many factors can be involved in alopecia, such as genetic factors, age, sex, diseases, stress, hormonal problems, side effects of medication, scars. Several forms of alopecia can be distinguished:
Hereditary androgenetic alopecia is the most frequent; early hair loss occurs in genetically predisposed individuals and affects men in particular. It is manifested by a decrease in hair volume, even baldness, and affects 50% of men over the age of 50.
Postmenopausal alopecia is the most common cause of baldness in women. Hair loss is more diffuse and extensive in women than in men. Diffuse female alopecia is a disorder that often starts at menopause and affects about 40% of women over the age of 70. The term diffuse illustrates that, unlike in men, hair loss affects the entire scalp, in a homogeneous manner.
Acute or reactive alopecia, which can be related to a drug treatment, stress, childbirth, significant nutritional deficiencies, iron deficiency, hormonal disorders, is a simultaneous and diffuse loss of a large quantity of hair.
Scarring alopecia can be caused by skin problems (tumor, burn, peeling), acute irradiation, lupus erythematosus or parasites (ringworm, lichen).
Alopecia areata seems to be of autoimmune origin and is characterized by a more or less large patchy attack in one or more places.
Congenital alopecia, which is rare, corresponds to an absence of root or to hair anomalies (mutations).

Alopecia is essentially linked to a disturbance in hair renewal which leads, initially, to an acceleration in the frequency of cycles at the expense of the quality of the hair and then of its quantity. The most frequent phenomenon is a reduction in the duration of the growth (anagen) phase in connection with a halt in cell proliferation. The consequence is a premature induction of the catagen phase and a greater number of hair follicles in the telogen phase and therefore greater hair loss. To combat alopecia, it is therefore necessary to restart the hair cycle, for example by activating the anagen phase.

To date, various products have been proposed to combat alopecia, and in particular to induce or stimulate hair growth. Most combine several active principles likely to have a beneficial effect on the biological parameters involved in hair loss. Among the most commonly encountered active principles, we can cite as examples: vitamins such as vitamins A, H, B5, B6, C, H and PP; trace elements such as zinc, copper, magnesium, silicon; protein derivatives such as peptides, sulfur-containing amino acids (such as methionine, cystine, cysteine or derivatives); essential oils or extracts of vegetable origin of lipophilic or hydrophilic nature, the list of which is non-limiting; antifungal agents such as piroctone olamine, undecyclinic derivatives, ciclopirox olamine; molecules of chemical synthesis known for their specific action at the level of the androgenic receptors or on the activity of the 5α-reductases. Minoxidil, or 2,4-diamino-6-piperidinopyrimidine-3-oxide, is today the reference in the treatment of androgenic alopecia. Despite the many theories on its mechanism of action, the latter is not clearly understood. Moreover, its effectiveness remains limited, because even if a stabilization of hair loss is observed in many clinical cases, a resumption of the alopecia process is observed as soon as the treatment is stopped. Its restrictive daily use is likely the source of undesirable side effects noted in patients using it on a long-term basis, such as localized skin reactions or systemic effects.

Furthermore, compositions comprising a wide range of active agents are proposed for hair regrowth, these active agents being for example 2,4-diaminopyrimidine-3-oxide derivatives such as those described in patent application EP0522964. Clinical studies have shown that PGF2α analogs have the property of inducing hair and eyelash growth in humans and animals (Johnstone, Am J Opht, 124(4), 544-547, 1997). In humans, tests carried out on the scalp have shown that a prostaglandin E2 analogue, viprostol, has the property of increasing hair density. Patent WO 98/33497 describes pharmaceutical compositions containing prostaglandins or prostaglandin derivatives intended to promote hair growth.

Thus, despite the many options currently available, consumers still need new products to promote hair regrowth that are natural and environmentally friendly, while being as effective as chemical active agents.

Recently, in experiments conducted on mouse and human hair follicles, researchers (Harel et al., Sci. Adv. 2015; e1500973) found that when applied to the skin, drugs that inhibit the Janus kinase (JAK) family of enzymes promote rapid and dense hair growth. Two JAR inhibitors are already approved by the Food and Drug Administration (FDA), one ruxolitinib for the treatment of blood diseases, the other tofacitinib for rheumatoid arthritis; both are the subject of clinical trials for alopecia areata. In these studies, it was noted that topical application of JAK inhibitors to the skin of mice resulted in hair regrowth, suggesting that in addition to blocking the autoimmune attack, they may have an effect on hair follicles, causing them to emerge from their telogen phase. Mice treated for 5 days with one of the two JAK inhibitors showed new hair regrowth within 10 days, demonstrating a rapid acceleration of the growth process, while no hair regrowth was observed in control mice over the same period.

Furthermore, Garcinia mangostana L. is a tropical tree native to Southeast Asia, now cultivated in many tropical countries for its edible fruit, the mangosteen, also called “fruit of the Gods” or “Queen of fruits”.

Garcinia mangostana A. is a dioecious tree that can reach 20 meters in height in the wild. Its leaves are smooth, shiny, leathery, elliptic to elliptic-oblong, measuring 14 to 25 cm by 5 to 10 cm. Their base is cuneiform to subrounded, the apex is short acuminate. The male flowers, rare, are in groups of 2 to 9 at the end of twigs, while the female flowers, a little larger than the male flowers, are solitary or in pairs. The fruit is made up of a thick pericarp of pink color to purplish very dark with maturity, containing an edible white flesh and enclosing 4 to 5 seeds.

Mangosteen has been known for centuries in traditional Asian medicine for its antioxidant, anti-inflammatory and antibacterial activities. The pericarp n particular is used in case of skin infection, diarrhea, abdominal pain, urinary tract infection, or bruises. It can also be used in case of fever (Ovalle-Magallanes et al., Food Chem Toxccol 109:102-122, 2017). There has also been a strong craze for manaosteen in the West over the past decade. Many fruit juices based on flesh and pericarp, dietary supplements and topical products containing mangosteen are marketed and recent clinical studies show their advantage in various indications such as weight loss (Udani et al., Nuts J, 8:48, 2009) and periodontitis (Mahendra et al., J Investig Clin Dent, 8(4), 2017). Mangosteen extracts are also found as active agents in several cosmetic products for their anti-aging or slimming properties.

Mangosteen pericarp is an important source of xanthones, the main ones being α-mangostin and γ-mangostin (Ovalle-Magallanes et al., 2017). In addition, mangosteen pericarp contains tannins, anthocyanins, and sugars. Fruit extracts, and more particularly mangosteen pericarp extracts more or less enriched in xanthones, have been the subject of numerous pharmacological studies, both in vitro and in vivo. The main activities shown are antitumor properties, especially in the prostate, lung, breast and colon, anti-inflammatory properties, antioxidant properties, anti-diabetic properties, anti-hyperlipidemic properties and antibacterial properties.

Mangosteen extracts in combination with other extracts, forming a specific composition for topical use, have been the subject of several patent applications in the field of hair science. Patent application JP2016069334 relates to a Garcinia mangostana extract in combination with hydrolyzed silk in liquid form, and positioned as a protective agent for hair against DV rays. Patent application CN103735453 relates to a composition for dyeing hair. Patent application US2006210515 relates to a topical composition comprising a partially hydrolyzed fucoidan, which may be useful in hair regrowth. Natural components can be added to this composition, such as mangosteen, but no activity in the field of hair science is associated therewith. A Thai study aimed at screening for plant extracts with 5α-reductase inhibitory activity, thus useful for treating benign prostate hypertrophy and/or genetic alopecia. An ethanolic extract of Garcinia mangostana pericarp came out of this screening but no relationship between the content of total phenolic compounds and 5α-reductase inhibitory activity could be established. The 5α-reductase inhibitory activity of Garcinia mangostana extracts has also been the subject of two patent applications. Application JP2000229857 attributes 5α-reductase inhibitory activity to xanthones, including α-mangostin. Xanthones can be extracted and isolated from plants of the families Hypericaceae (Guttiferae), Gentianaceae, Mulaceae, Polygalaceae, Liliaceae, and Plumaceae. The preparation of a benzene extract of Garcinia mangostana L. is described. Application JP5017365 relates to several plant extracts including aqueous extracts or extracts obtained by hydrophilic organic solvents, such as for example a methanolic extract, of Garcinia mangostana and can be formulated for topical application.

Thus, to date no bibliographic data mention that a Garcinia mangostana L. extract can have an activity in hair regrowth.

### SUMMARY OF THE INVENTION

Surprisingly, the inventors have discovered that a Garcinia mangostana L. extract has pharmacological activities of interest for promoting hair growth, particularly for promoting hair regrowth by acting on this JAK target. These activities are detailed in Examples 11 to 13.

The invention therefore relates to the treatment of hair loss insofar as the Garcinia mangostana L. extract acts on the functions or biological mechanisms at the origin of hair growth. More particularly, the invention relates to a hydroalcoholic extract of Garcinia mangostana and a dermatological composition comprising said extract with at least one dermatologically acceptable excipient for use in the prevention or treatment of alopecia by promoting hair growth.

The invention also relates to the non-therapeutic use of a hydroalcoholic extract of Garcinia mangostana to promote hair regrowth. The invention relates to a cosmetic composition for promoting hair regrowth, in particular for promoting hair regrowth comprising at least one hydroalcoholic extract of Garcinia mangostana with at least one dermatologically acceptable excipient.

### DEFINITIONS

In the present invention, the plant Garcinia mangostana L. may be abbreviated by the term Garcinia mangostana.

“Garcinia mangostana extract” means the extraction product of all or part of the Garcinia mangostana plant.

“Extraction product” means the product obtained after extraction of a part of the plant, with a solvent, called extraction solvent, i.e., a product present in the extraction solvent which can then possibly be in a concentrated or dry form after partial or total evaporation of the extraction solvent. It can be a dry extract.

“Dry extract” means, in the sense of the present invention, an extract without extraction solvent or carrier, or containing only an insignificant trace amount of it. Such a dry extract thus contains only material from Garcinia mangostana. It may also contain insignificant trace amounts of extraction solvent.

In the present invention, “about” means that the value concerned may be 10%, in particular 5%, in particular 1%, lower or higher than the value indicated. This definition applies in particular when defining the content by weight of alpha-mangostin relative to the weight of the dry extract.

“Hydrophilic solvent” means a solvent selected from the group consisting in particular of water, subcritical water, water-miscible alcohols such as ethanol, C3 to C5 glycols, glycerol, acetone, and mixtures thereof.

“Apolar solvent” means, in the sense of the present invention, a solvent selected for example from heptane, hexane, limonene, halogenated hydrocarbons (chloroform, dichloromethane), supercritical CO₂, a mixture of supercritical CO₂ and ethanol.

“Moderately polar solvent” means, in the sense of the present invention, a solvent selected from the group consisting in particular of C1 to C5 alcohols, glycols such as propylene glycol, butylene glycol, butanediol or pentylene glycol, glycerol, acetone, alkyl esters such as ethyl acetate, isopropyl acetate, water-miscible solvents (a hydroalcoholic mixture or an acetone/water mixture, for example). Also included in this group are alternative solvents of the hydrotropic type (amphiphilic molecules soluble in water and which, from a sufficient concentration, can extract moderately polar compounds as described in the characterization of the extract).

“Head and body hair” means head hair, body hair, eyebrows, eyelashes and/or fur, preferentially hair.

“Skin appendages” means head hair, body hair, eyebrows, eyelashes, and/or nails, preferentially hair.

“Alopecia” means the total or partial loss of head hair and/or body hair, for example due to reduced hair growth and/or accelerated hair loss. This term includes but is not limited to androgenetic alopecia, postmenopausal alopecia, reactive alopecia, scarring alopecia, alopecia areata, and congenital alopecia. The consequences of alopecia are a temporal or permanent and partial or total absence of head hair and/or body hair.

“Treat” alopecia means to stop alopecia, reduce alopecia and/or alleviate alopecia. Thus, “treating” alopecia includes limiting head hair and/or body hair loss and/or promoting head hair and/or body hair growth, increasing hair follicle density and/or regulating the phases of the hair follicle cycle.

“Prevent” alopecia means to reduce the risk of alopecia appearing, or to slow the progression of alopecia in a mammal, preferably a human, who is likely to develop alopecia.

“Limit” means to slow, reduce, decrease and/or stop.

“Promote” means to increase, enhance, favor, amplify and/or accelerate.

In the present invention, “cosmetically or dermatologically acceptable” means that which is useful in the preparation of a cosmetic, dermatological composition, which is generally safe, non-toxic and neither biologically nor otherwise undesirable and which is acceptable for cosmetic or dermatological use, particularly by topical application to the hair and/or scalp.

“Topical application” means an application to the skin, in particular to the scalp, the mucous membranes, and/or the skin appendages, in particular to the hair and the scalp.

### DETAILED DESCRIPTION OF THE INVENTION

According to a first aspect, the invention relates to a Garcinia mangostana extract for use in promoting hair growth, more particularly for promoting hair regrowth.

In the context of the present invention, the Garcinia mangostana extract is obtained from one or more parts of the Garcinia mangostana plant selected from aerial parts such as fruit, fruit pericarp, fruit pulp, seeds, leaves, stems and/or bark.

In a particular embodiment of the invention, the extract is obtained from the fruits and/or pericarps of the Garcinia mangostana fruit. Advantageously, the extract is obtained from the pericarp of the Garcinia mangostana fruit.

In a particular embodiment of the invention, the extract is obtained from a culture of Garcinia mangostana cells.

The extract is a hydroalcoholic extract, in particular hydroethanolic extract.

According to a preferred embodiment, the extract according to the invention is obtainable by a process as described below.

The Garcinia mangostana plant or plant part can be fresh or dry, whole, cut or ground and then subjected to an extraction step.

A process for preparing an extract according to the invention comprises a step of extracting all or part of the Garcinia mangostana plant with an alcohol/water mixture.

In an embodiment of the invention, the extraction solvent is an alcohol/water mixture, wherein the alcohol may be a C3 to C5 glycol or a C1 to C5 alcohol.

Advantageously, the alcohol water mixture is characterized by an alcohol/water ratio of 9:1 to 7:3 (v/v). Even more advantageously, the alcohol/water mixture is characterized by an alcohol/water ratio of 9:1 (v/v).

Advantageously, it will be an ethanol/water mixture.

Even more advantageously, this ethanol/water mixture will be characterized by an ethanol/water ratio of 9:1 to 7:3 (v/v).

And even more advantageously, this ethanol/water mixture will be characterized by an ethanol/water ratio of 9:1 (v/v).

According to another particular embodiment of the invention, the extraction is carried out under stirring or statically, at reflux, at room temperature, or at a temperature between room temperature and reflux. It can be assisted by ultrasound, microwave, flash expansion or extrusion, in a plant weight to solvent volume ratio that can vary from 1:3 to 1:30, for a period of 1 minute to 48 hours. The extraction can be repeated 2 to 3 times.

According to another particular embodiment of the invention, the pomace is then separated from the extract by centrifugation or filtration in order to recover a clear liquid phase free of particles. The liquid phase representing the extract can be more or less concentrated up to the point of obtaining a dry extract.

In another embodiment of the invention, a carrier may be added during the concentration step so as to obtain an extract containing 1 to 75% dry extract. The carrier may be maitodextrin, lactose, silica, glycerine, a glycol, a vegetable oil, a hydrotrope, a water/solubilizer or water/surfactant mixture, or any other cosmetically acceptable carrier which solubilizes the extract, preferably of biosourced origin, such as for example biosourced glycols (1,2-pentanediol; 1,3-butanediol; 1,3-propanediol; etc.), esterified fatty acids and also hydrotropes such as alkyl glycosides (Sepiclear, Apyclean, APXC4, etc.).

According to a particular embodiment of the invention, the Garcinia mangostana extract can be decolorized, for example on activated carbon. Advantageously, the Garcinia mangostana extract is not decolorized.

The hydroalcoholic extract of Garcinia mangostana according to the present invention induces inhibition of the JAK-STAT signaling pathway. Furthermore, the hydroalcoholic extract of Garcinia mangostana according to the present invention inhibits melanin synthesis.

According to a second aspect, the present invention relates to a dermatological or cosmetic composition comprising at least one Garcinia mangostana extract with at least one dermatologically or cosmetically acceptable excipient for use in promoting hair growth, more particularly for promoting hair regrowth.

Advantageously, the extract comprised in the dermatological or cosmetic composition is as described above.

According to a particular embodiment of the invention, the cosmetic or dermatological composition comprising at least one Garcinia mangostana extract as described above and at least one cosmetically or dermatologically acceptable excipient comprises from 0.001 to 5% by weight, more preferably from 0.002 to 2% by weight, more preferably from 0.005 to 1% by weight, still more preferably from 0.01 to 0.5% of Garcinia mangostana extract, by weight of dry extract relative to the total weight of the composition.

The invention is preferably directed to a cosmetic or dermatological composition according to the invention which is in a form suitable for topical application, in particular to the scalp and/or hair.

The cosmetic or dermatological composition according to the invention can thus be in the forms which are usually known for topical administration, i.e., in particular lotions, shampoos, balms, foams, gels, dispersions, emulsions, sprays, serums, masks or creams, with excipients which allow in particular penetration in order to improve the properties and accessibility of the active principle.

Advantageously, the composition according to the invention may be in the forms that are usually known for topical administration to the hair and scalp, i.e., in particular a shampoo, conditioner, hair cream, hair lotion, mask or leave-in spray.

A distinction is made between formulated products that can be rinsed and formulated products that do not require rinsing.

Preferably, the composition according to the invention has a light texture further allowing an optimal penetration without making the head hair and/or body hair, nor the scalp, greasy.

In a particular embodiment of the invention, the composition according to the invention is characterized in that it is in a form suitable for oral administration.

According to another embodiment of the invention, the composition according to the invention may also be in the forms that are commonly known for oral administration, i.e., in particular tablets, capsules, powders, granules and oral solutions or suspensions. When preparing a solid composition in tablet form, the principal active ingredient is mixed with a pharmaceutical carrier such as gelatin, starch, lactose, magnesium stearate, talc, gum arabic, silica or the like. The tablets may be coated with sucrose or other suitable materials or they may be processed in such a way that they have an extended or delayed activity and continuously release a predetermined amount of active principle.

A capsule preparation can be obtained by mixing the active ingredient with a diluent and pouring the resulting mixture into soft or hard capsules.

The composition according to the invention, administered for example topically or orally, generally contains, in addition to the extract as described above, a physiologically acceptable medium, generally water- or solvent-based, for example alcohols, ethers or glycols. It may also contain surfactants, complexing agents, preservatives, stabilizers, emulsifiers, thickeners, gelling agents, humectants, emollients, trace elements, essential oils, perfumes, dyes, moisturizing agents or geothermal water, etc.

From the very first administrations, for example topically or orally, of the composition according to the invention, the hair will regain strength and vitality.

According to the first or second aspect of the invention, the extract as described above or the composition as described above can thus be used to promote hair growth, more particularly to promote hair regrowth.

In a particular embodiment, the Garcinia mangostana extract as described above is the sole active principle for promoting hair growth, more particularly for promoting hair regrowth.

According to a particular embodiment of the invention, the Garcinia mangostana extract as described above is characterized by a content of 0.5 to 80%, particularly 5 to 80%, more particularly 10 to 60%, or even more particularly about 15% by weight of alpha-mangostin relative to the weight of the dry extract.

According to an embodiment of the invention, the Garcinia mangostana extract as described above or the cosmetic or dermatological composition comprising at least one Garcinia mangostana extract and at least one cosmetically or dermatologically acceptable excipient as described above, is intended to be applied topically and/or orally, preferably topically.

The cosmetic or dermatological extract or composition as described above may be used in an individual who has undergone a hair micrograft.

According to a particular embodiment of the invention, the Garcinia mangostana extract as described above or the composition as described above prevents or treats alopecia, which may be selected from the group consisting of androgenetic alopecia, reactive alopecia, postmenopausal alopecia and alopecia areata.

The object of the invention is a Garcinia mangostana extract or a dermatological composition comprising at least one Garcinia mangostana extract with at least one dermatologically acceptable excipient, as defined above, for use in the prevention and/or treatment of alopecia, said alopecia which can be selected from the group consisting of reactive alopecia and alopecia areata.

Another object of the invention is the use of a Garcinia mangostana extract or a dermatological composition comprising at least one Garcinia mangostana extract with at least one dermatologically acceptable excipient, as defined above, for manufacturing a pharmaceutical or dermatological composition intended for promoting hair growth, in particular for preventing and/or treating alopecia, for example reactive alopecia and alopecia areata.

The invention is also directed to the use of a Garcinia mangostana extract or a dermatological composition comprising at least one Garcinia mangostana extract with at least one dermatologically acceptable excipient, as defined above, for promoting hair growth, most particularly for promoting hair regrowth, in particular for preventing and/or treating alopecia, for example reactive alopecia and alopecia areata.

The invention is also directed to a method for promoting hair growth, in particular for preventing and/or treating alopecia, for example reactive alopecia and alopecia areata, comprising administering to a patient in need thereof an effective amount of a Garcinia mangostana extract or a cosmetic or dermatological composition comprising at least one Garcinia mangostana extract with at least one cosmetically or dermatologically acceptable excipient, as defined above.

The invention is also directed to a cosmetic (non-therapeutic) use of the extract as described above according to a previously described embodiment or of the cosmetic or dermatological composition as described above according to a previously described embodiment, for hair and/or scalp care, and/or for promoting hair growth, more particularly for promoting hair regrowth, and/or for increasing the density of the hair follicles and/or for obtaining more covering hair and/or for promoting follicular regeneration and/or for combating alopecia, it being possible for said alopecia to be selected from androgenetic alopecia and post-menopausal alopecia.

The invention also relates to a non-therapeutic cosmetic method for hair and/or scalp care, and/or for promoting hair growth, more particularly for promoting hair regrowth, and/or for increasing the density of the hair follicles and/or for obtaining more covering hair and/or for promoting follicular regeneration and/or for combating alopecia, said alopecia which can be selected from androgenetic alopecia and post-menopausal alopecia.

Another object of the invention is a non-therapeutic cosmetic method for promoting hair growth, more particularly for promoting hair regrowth, in particular for preventing and/or treating alopecia, for example androgenetic alopecia and postmenopausal alopecia, comprising the use of a Garcinia mangostana extract or of a cosmetic or dermatological composition comprising at least one Garcinia mangostana extract with at least one cosmetically or dermatologically acceptable excipient, as defined above.

### EXAMPLES

The following examples illustrate the invention without limiting its scope.

The first ten examples relate to the preparation of an extract used for the invention.

### Example 1: Reflux Extraction With 90% Ethanol

377 grams of ground dry Garcinia mangostana pericarps is extracted under reflux with stirring with 3.8 liters of a 90:10 (v/v) ethanol/water mixture for 1 hour in a reactor. The extract is then filtered on a K900 filtration plate and the solvent is evaporated to obtain 100 grams of an orangish powder with a mass yield of 26%. The dry extract obtained contains 15.9% by weight of alpha-mangostin.

### Example 2: Reflux Extraction With 90% Ethanol Followed by Support

200 grams of ground dry Garcinia mangostana pericarps is extracted under reflux with stirring with 2 liters of a 90:10 (v/v) ethanol/water mixture for 1 hour in a reactor. The extract is then filtered on a K900 filtration plate and dried on maltodextrin to obtain 142 grams of extract as an orange-beige powder. The extract contains 75% maltodextrin and 3.5% alpha-mangostin by weight of the extract.

### Example 3: Reflux Extraction With 90% Ethanol Followed by Support

1000 grams of ground dry Garcinia mangostana pericarps is extracted under reflux with stirring with 10 liters of a 90:10 (v/v) ethanol/water mixture for 1 hour in a reactor. The extract is then filtered on a K900 filtration plate and dried on 1,2-pentanediol so as to obtain 747 grams of extract in the form of a dark viscous liquid. The extract contains 70% 1,2-pentanediol and 4.9% alpha-mangostin by weight of the extract.

### Example 4: Reflux Extraction With Hexane

17 grams of ground dry Garcinia mangostana pericarps is extracted under reflux with 170 milliliters of hexane for 1 hour in a reactor. The extract is then filtered on a K900 filtration plate and the solvent is evaporated so as to obtain 200 milligrams of an orange-yellow paste with a mass yield of 1.2%. The dry extract obtained contains 75.0% by weight of alpha-mangostin.

### Example 5: Ultrasound-Assisted Extraction With 96% Ethanol

36 grams of ground dry Garcinia mangostana pericarps is put in contact with 350 milliliters of 96% ethanol and then extracted under the action of ultrasound (20 kHz) for 3 times one minute at an amplitude of 100%. The extract is then filtered on a K900 filtration plate and the solvent is evaporated so as to obtain 7.9 grams of a purplish-red powder with a mass yield of 22%. The dry extract obtained contains 16.4% by weight of alpha-mangostin.

### Example 6: Reflux Extraction With 96% Ethanol

20 grams of ground dry Garcinia mangostana pericarps is extracted under reflux with 200 milliliters of 96% ethanol for 1 hour in a reactor. The extract is then filtered on a K900 filtration plate and the solvent is evaporated to obtain 5.3 grams of a purplish-red powder with a mass yield of 26%. The dry extract obtained contains 16.0% by weight of alpha-mangostin.

### Example 7: Hydrotropic Extraction With a 1.5 19 Aqueous Heptylglucoside Solution

26 grams of ground dry Garcinia mangostana pericarps is extracted for 2 hours at 40° C. with stirring with 260 milliliters of a 1.5 M aqueous heptylglucoside solution. After filtration on a K900 filtration plate, the filtrate is acidified to pH=2 and then diluted with 11 volumes of acidified water at pH=2. After centrifugation, the pellet is collected and dried so as to obtain an orangish paste with a mass yield of 7.3%. The dry extract obtained contains 17.0% by weight of alpha-mangostin.

### Example 8: Hydrotropic Extraction With a 25% Aqueous Butyl Xyloside Solution

20 grams of ground dry Garcinia mangostana pericarps is extracted for 2 hours at room temperature with stirring with 200 milliliters of a 25% mass aqueous butyl xyloside solution. After filtration on a K900 filtration plate, the filtrate is diluted with 2 volumes of water. After centrifugation, the pellet is recovered. The supernatant is diluted with 2 volumes of water. After centrifugation, the pellet is recovered and combined with the previous pellet, so as to obtain the dry extract of Garcinia mangostana in the form of a brown powder with a mass yield of 4.9%. The dry extract obtained contains 53.0% by weight of alpha-mangostin.

### Example 9: Extrusion-Assisted Hydrotropic Extraction With an Aqueous Butyl Xyloside Solution (APXC4)

670 grams of dry Garcinia mangostana pericarp is introduced into the first barrel of a Clextral BC45 twin-screw extruder with a flow rate of 40 kg/h. A 26% by weight aqueous butyl xyloside solution is then introduced at a flow rate of 120 kg/h. The temperature applied to the different barrels is 60° C. After one minute, the extract of Garcinia mangostana pericarp is recovered at the extruder outlet through a filtering barrel allowing a solid/liquid separation. After clarification, the solution is diluted with 2 volumes of water. After centrifugation, the pellet is recovered. The supernatant is diluted with 2 volumes of water. After centrifugation, the pellet is recovered and combined with the previous pellet, in order to obtain the dry extract of Garcinia mangostana in the form of orangish paste with a mass yield of 0.4%. The dry extract obtained contains 50.0% by weight of alpha-mangostin.

### Example 10: Extraction With Supercritical CO₂

470 grams of ground dry Garcinia mangostana pericarps is extracted for 2 hours at 50° C. and 50 bar with supercritical CO₂ with a flow rate of 10 kg/h. The extract is then solubilized in ethanol and filtered on a K900 filtration plate and the solvent is evaporated to obtain 3.4 grams of an orange-yellow paste with a mass yield of 0.7%. The dry extract obtained contains 9.45% by weight of alpha-mangostin.

The pomace (466 grams) is then extracted for 1 hour at 50° C. and 50 bar with supercritical CO₂ with ethanol as co-solvent (flow rates of 10 kg/h and 1 kg/h, respectively). The extract is filtered on a K900 filtration plate and the solvent is evaporated to obtain 2.3 grams of an orange-yellow paste with a mass yield of 0.5%. The dry extract obtained contains 22.3% by weight of alpha-mangostin.

### Example 11: Evaluation of a Garcinia mangostana Extract on the Inhibition of Human JAK1, JAK2 and JAK3

JAK-STAT is a transduction signaling pathway regulating growth, survival, differentiation and pathogen resistance. This pathway mediates the effects of cytokines, interferons and growth factors. In mammals, the JAK family consists of four members: JAK1, JAK2, JAK3 and TYK2. It has been shown in a study performed on mouse hair follicles that the JAK-STAT pathway is dynamically regulated in the hair cycle; indeed, the JAK-STAT pathway is activated during the catagen and telogen phases and repressed at the beginning of the anagen phase. Moreover, it has been shown in mice that topical treatment in telogen phase with inhibitors of the JAK-STAT pathway, including tofacitinib (JAK1/3>JAK2>TYR2) and ruxolitinib (JAK1/2>TYR2>JAK3), resulted in rapid re-entry at the beginning of anagen phase (Harel et al., 2015 Sci. Adv. 1, e1500973). In this same study it is also shown that inhibition of JAK-STAT in human hair follicles increases hair growth ex vivo. These data suggest that the JAK-STAT signaling pathway may be a new target for stimulating hair growth.

The purpose of this example is to evaluate whether a Garcinia mangostana extract could inhibit the JAK-STAT signaling pathway. This inhibition is evaluated on recombinant human JAK1, JAK2 or JAK3 proteins.

### Methods

A Garcinia mangostana extract according to Example 1 is diluted in DMSO and tested at various concentrations (0.1-1000 μg/ml). Positive controls (tofacitinib and ruxolitinib) are also evaluated in this test.

The test products are incubated with recombinant human JAK1, JAK2 or JAK3 protein with a reaction buffer (Tris/HCl for JAK1, MOPS (3-morpholino-l-propanesulfonic acid) for JAK2 and JAK3), EDTA and the specific peptide substrates. The phosphorylation reaction is then initiated by the addition of a mixture of magnesium acetate and radiolabeled ATP (45 μM for JAK1 and JAK2 and 10 μM for JAK3). After incubation for 40 minutes at room temperature, the reaction was stopped by adding phosphoric acid. Four washes with phosphoric acid and one with methanol are performed to elute the small molecules including the labeled ATP. Finally, the radioactivity of the specific phosphorylated substrate is counted. The compounds are tested on 3 distinct experiments and for each experiment, duplicates are performed.

Inhibition curves are constructed and IC₅₀ values are calculated for each JAK subtype. An IC₅₀ (50% inhibitory concentration) is the concentration of a compound that inhibits 50% of the observed effect.

The IC₅₀ values, in μg/ml, of each recombinant JAK1, JAK2 and JAK3 protein as a function of the incubated test product are presented in the following Table 1:

TABLE 1

| | JAK1 | JAK2 | JAK3 |
|---|---|---|---|
| Compounds | IC₅₀ (μg/ml) | IC₅₀ (μg/ml) | IC₅₀ (μg/ml) |
| Ruxolitinib | 0.00037 | 0.00023 | 0.0060 |
| Tofacitinib | 0.00087 | 0.00367 | 0.00057 |
| Gar mang extract | 6.6 | 46.2 | 1.4 |

Gar mang: Garcinia mangostana

The results obtained with the reference compounds (ruxolitinib and tofacitinib) are consistent with those expected. Indeed, both compounds strongly inhibit JAK-STAT activity (between 97% and 100% maximal inhibition, with very low IC₅₀ values (in the nanogram per milliliter range). Ruxolitinib shows a higher affinity for JAK1 or JAK2 than for JAK3, while tofacitinib has more affinity for JAK1 or JAK3 than for JAK2. These expected results validate this test.

The Garcinia mangostana extract shows a strong inhibition of JAK1 (100% maximal inhibition) with an IC₅₀ value of 6.6 μg/ml, a strong inhibition of JAK2 (100% maximal inhibition) with a slightly higher IC₅₀ value of 46.2 μg/ml, and also a strong inhibition for JAK3 (100% maximal inhibition) with an IC₅₀ value of 1.4 μg/ml.

This test shows that the Garcinia mangostana extract induces an inhibition of tyrosine kinase activity (with a stronger affinity for JAK1 and JAK3 than for JAK2), revealing a pharmacological activity of interest for promoting hair growth.

### Example 12: Confirmation of the JAK-STAT Pathway Inhibitory Activity of a Garcinia mangostana Extract at the Cellular Level

The purpose of this example is to confirm the JAK-STAT signaling pathway inhibitory activity of a Garcinia mangostana extract on a cellular model, the follicular keratinocytes of the outer epithelial sheath of hair follicles (outer root sheath, or ORS, model). In this model, interleukin IL-6 is used to activate the JAK1/2-STAT3 pathway through the activation of IL-6 and GP130 receptors, both of which are expressed in this hair follicle epithelial sheath. IL-6 is a cytokine that acts as an inhibitor of the hair growth cycle; its overexpression in a transgenic mouse model results in delayed hair growth. Furthermore, in androgenetic alopecia IL-6 is reported to be overexpressed in dermal papilla cells under the influence of androgens (Kwack et al., 2012, J Invest Dermatology 132(1) 43-9). It has also been reported that IL-6 delays hair follicle growth in humans.

### Method

The study is performed on follicular keratinocytes from the outer epithelial sheath of hair follicles (outer root sheath, or ORS, model). Keratinocytes are cultured in 96-well plates in a suitable medium (CnTa-PR from Cellntec). Twenty-four hours later, the cells are washed with alkaline phosphate buffer (PBS) and the medium is changed to CnT-PR-H medium (standard maintenance medium for primary human keratinocytes). After 48 hours, the cells are treated for 1 hour with the test compounds (Garcinia mangostana extract at 10 and 30 μg/ml diluted in DMSO) and the reference compounds (ruxolitinib and tofacitinib both at 5 μM diluted in DMSO). The Garcinia mangostana extract tested is an extract according to Example 1 of the present invention. Then the IL-6 stimulation treatment is performed at 100 ng/ml for 15 minutes.

All conditions are tested on cells from n=3 distinct donors. The Garcinia mangostana extracts are tested on n=3 distinct experiments, the control conditions, of stimulation and with the reference compounds are performed on n=6 experiments. For each experiment, the data are performed in triplicate. JAK activity is estimated by evaluating the level of phosphorylation of the STAT3 protein. Statistical analysis is performed by a parametric test after checking normality and equivalence of variance, otherwise a non-parametric test is selected.

### Results

The results are summari ed in the following Table 2:

TABLE 2

Level of
phosphorylated
STAT3

| Condition | Groups | Mean | SEM | % inh |
|---|---|---|---|---|
| — | Control | 1.11 | 0.04 | 100 |
| IL-6 | Control | 1.32 | 0.03 | 0 |
| IL-6 | Ruxolitinib | 1.13 | 0.05 | 90 |
| IL-6 | Tofacitinib | 1.20 | 0.05 | 59 |
| IL-6 | Garc mang (10 μg/ml) | 0.92 | 0.04 | 190 |
| IL-6 | Garc mang (30 μg/ml) | 1.03 | 0.03 | 136 |

SEM: standard error of the mean; % inh: percentage of inhibition; Garc mang: extract of Garcinia mangostana.

Treatment of keratinocytes with IL-6 induced a significant increase in the level of STAT3 phosphorylation, this increase reached 19% and was statistically significant (p<0.01). The reference compounds (ruxolitinib and tofacitinib tested at 5 μM) significantly reduce the level of STAT3 phosphorylation, respectively by 90% and 59%, p<0.05 for both compounds. These expected results validate the test.

The Garcinia mangostana extract at the two concentrations tested significantly and in a very marked way inhibits the level of STAT3 phosphorviation induced by IL-6 stimulation.

The inventors thus demonstrated the advantage of a Garcinia mangostana extract in the promotion of hair regrowth.

### Examples 13: Evaluation of a Garcinia mangostana Extract on Versican Synthesis and Release

The anchoring of hair follicles results from a complex organization, composed of proteoglycans, matrix proteins such as collagen and anchoring structures such as desmosomes and hemidesmosomes. Proteoglycans are involved in cell adhesion to the extracellular matrix, cell-to-cell adhesion, as well as cell differentiation. Desmosomes also mediate cell-cell adhesion and allow anchoring of the intermediate filament network to the plasma membrane. A study shows that mutations in hair follicle anchoring proteins and more specifically in desmosome proteins results in hypotrichosis in mice but also in humans (Nagasawa et al., Dermatol Ther (Heidelb), 2016, 6:59-68). Hypotrichosis is a disease of both the skin and scalp that refers to an arrest of all hair growth. In addition, it is reported that androgenetic alopecia is associated with miniaturization of the hair follicle and ultimately a loss of anchoring properties.

Proteoglycans are composed of the association of glycosaminoglycans, which are anionic polysaccharides, and basic proteins. Each basic protein is preferentially associated with a specific glycosaminoalvcan chain. Glycosaminoglycans are classified as chondroitin sulfate, heparan sulfate, keratin sulfate or dermatan sulfate according to their chemical structure. It has been shown that there is a variable distribution of the components of these proteoglycans depending on the location and growth state of the hair. The dermal papilla contains an high level of basic protein and a wide variety of glycosaminoglycans. These glycosaminoglycans are known to bind and modulate a large number of biomolecules involved in cell differentiation or proliferation. Mention may be made of fibroblast growth factor (FGF), vascular endothelial growth factor (VEGF), sonic hedgehog (Shh), bone morphogenetic protein (BMPs), wingless integration site (Ent) and hepatocyte growth factor (HGF). All these factors are well known to be involved in hair follicle morphogenesis. This network by forming a microenvironment specific to the hair follicle also acts as a reservoir of modulators and growth factors and is therefore involved in the homeostasis of the hair follicle and in the regulation of the proliferation and differentiation of follicular cells.

Versican is a large proteoglycan of the chondroitin sulfate family. It is produced by fibroblasts, smooth muscle cells and keratinocytes and is involved in skin maintenance and firmness. It is involved in cell adhesion within the extracellular matrix. It plays a significant role in cell migration, proliferation and differentiation. Versican is expressed in the dermal papilla of the hair follicle, in the proximal part of the connective tissue sheath, with a progressive decrease towards the distal part. The expression of its specific gene in the dermal papilla is high during the anagen phase and decreases at the beginning of the catagen phase. Its expression is regulated by the β-catenin signaling pathway. This specific expression of versican shows its importance in the hair growth phase.

The purpose of this example is to evaluate whether a Garcinia mangostana extract can influence the synthesis and release of versican, an anchoring component of the hair follicle.

### Method

Experiments are performed on human dermal papilla cells derived from the hair follicles of three donors. The cells are seeded in 96-well plates and cultured for 24 h with the necessary supplements. The cells are then treated for 48 h with the test products (Garcinia mangostana extract at 3 or 10 μg/ml diluted in DMSO). The Garcinia mangostana extract tested is an extract according to Example 1 of the present invention. At the end of the incubation, the supernatants are collected and an ELISA is performed to evaluate the synthesis and release of versican. All experimental conditions are performed with n=3 donors and n=3 technical replicates. The amount of versican in the supernatants is measured with a specific ELISA kit according to the instructions of the supplier (Cusabio).

### Results

Versican concentrations measured the supernatants are represented in Table 3 below:

TABLE 3

| | Mean concentration | | |
|---|---|---|---|
| | of versican in the | | |
| | supernatant (ng/ml) | SEM | Stats |
| Control | 16.71 | 2.65 | — |
| Garcinia mangostana | 15.56 | 2.39 | p = NS |
| extract at 3 μg/ml | | | |
| Garcinia mangostana | 46.61 | 11.32 | p < 0.01 |
| extract at 10 μg/ml | | | |

SEM: standard error of the mean; Stats: the inter-group comparison is performed by a repeated measures ANOVA followed by a Dunnett's post-hoc test.

Treatment of dermal papilla cells with Garcinia mangostana extract at 10 μg/ml also induces a strong statistically significant (p<0.01) activation of versican synthesis and release. A lower concentration of Garcinia mangostana extract at 3 μg/ml is not sufficient to activate this target. The Garcinia mangostana extract inhibits JAK-STAT activity with values in the range of 1 to 46 μg/ml depending on the subtype as shown in Example 10. It is therefore not illogical that a concentration higher than 3 μg/ml is required to significantly increase the synthesis and release of a proteoglycan since the activation of versican is not related to the inhibition of the JAK/STAT pathway.

1-13. (canceled)

14. A method of preventing or treating alopecia by promoting hair growth, the method comprising administering to a subject in need thereof an effective amount of a hydroalcoholic extract of Garcinia mangostana.

15. The method according to claim 14, wherein said extract is a pericarp extract of the fruit of Garcinia mangostana.

16. The method according to claim 14, wherein said extract is intended to be applied topically.

17. The method according to claim 14, wherein the alopecia is selected from the group consisting of androgenetic alopecia, reactive alopecia, postmenopausal alopecia, and alopecia areata.

18. A non-therapeutic method for promoting hair regrowth, the method comprising administering to a subject in need thereof an effective amount of a hydroalcoholic extract of Garcinia mangostana.

19. A method of preventing or treating alopecia by promoting hair growth, the method comprising administering to a subject in need thereof an effective amount of a dermatological composition comprising at least one hydroalcoholic extract of Garcinia mangostana and at least one dermatologically acceptable excipient.

20. The method according to claim 19, wherein the Garcinia mangostana extract is an extract of the pericarp of the Garcinia mangostana fruit.

21. The method according to claim 19, wherein the dermatological composition comprises 0.001 to 5% of Garcinia mangostana extract, by weight of dry extract relative to the total weight of the composition.

22. The method according to claim 19, wherein the composition is intended for topical application.

23. The method according to claim 19, wherein the alopecia is selected from the group consisting of androgenetic alopecia, reactive alopecia, postmenopausal alopecia, and alopecia areata.

24. A cosmetic composition for promoting hair growth comprising at least one hydroalcoholic extract of Garcinia mangostana with at least one dermatologically acceptable excipient.

25. The cosmetic composition according to claim 24, wherein the hydroalcoholic extract of Garcinia mangostana is the sole active agent for promoting hair growth.

26. A non-therapeutic method for promoting hair growth, the method comprising administering to a subject in need thereof an effective amount of a cosmetic composition comprising at least one hydroalcoholic extract of Garcinia mangostana and at least one cosmetically acceptable excipient.

27. The method according to claim 19, wherein the dermatological composition comprises 0.005 to 1% of Garcinia mangostana extract, by weight of dry extract relative to the total weight of the composition.

28. The cosmetic composition according to claim 24, wherein the cosmetic composition is for promoting hair regrowth.

29. The cosmetic composition according to claim 28, wherein the hydroalcoholic extract of Garcinia mangostana is the sole active agent for promoting hair growth.
